(19) United States
(12) Patent Application Publication
(10) Pub. No.: US 2021/0068492 A1
(43) Pub. Date: Mar. 11, 2021

(54) Footwear and Method

(71) Applicant: Emmanuel Onyekachi, avondzle, AZ (US)

(72) Inventor: Emmanuel Onyekachi, avondzle, AZ (US)

(21) Appl. No.: 17/016,293

(22) Filed: Sep. 9, 2020

Related U.S. Application Data
(60) Provisional application No. 62/897,856, filed on Sep. 9, 2019.

(51) Int. Cl. A43B 3/00; A43B 1/00; A43B 7/00; G01G 19/52; G01C 22/00; G01G 23/37

(52) U.S. Cl. CPC A43B 3/0005; A43B 1/0054; G01G 23/3742; G01G 19/52; G01C 22/006; A43B 7/00

(57) ABSTRACT

Footwear with a weight sensor for determining the weight of the user, a step counter for determined the number of steps a user takes, a display to show weight and steps, and a wireless transceiver to communicate with a smartphone or smartwatch. The footwear can be hung using the integrated magnet.

### CROSS-REFERENCE TO RELATED APPLICATION(S)

The present application is related to and claims priority to U.S. Provisional Patent Application No. 62/897,856 filed Sep. 9, 2019, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is expressly or implicitly referenced is prior art.

### TECHNICAL FIELD

The present invention generally relates to the field of footwear.

### RELATED ART

Individuals with medical conditions often need to be aware of changes in their weight. However, many individuals are either not diligent about checking their weight or do not own a scale. For some, the only time they see their weight is when they visit the hospital or doctor's office. Medical attention may be required for some individuals based on their weight.

U.S. Pat. No. 5,815,954 to Tien-Tsai Huang relates to a shoe with an electronic step counter. The described shoe with an electronic step counter includes a shoe with an electronic step counter, including a shoe body, a fluid bladder, a pressure sensor, a temperature compensator, a transmitting circuit, and a receiving circuit. The shoe body has a vamp and an outsole having a receiving space for receiving the fluid bladder. The pressure sensor contacts the fluid in the fluid bladder to detect the pressure thereof and generates a pressure voltage signal. The temperature compensator communicates with the fluid in the fluid bladder, detects the temperature thereof, and generating a temperature voltage signal. The transmitting circuit is disposed in the receiving space of the outsole and connected to the pressure sensor and the temperature compensator. The transmitting circuit receives the pressure voltage signals and the temperature voltage signals, convert them into a value that is emitted in a radio signal. The receiving circuit is disposed to separate from the transmitting circuit and may receive the radio signal from therefrom. The receiving circuit demodulates and decodes the received radio signal and displays the thus obtained value.

US Pub. No. 20090038182 to Maris J. Lans and Margarita V. Lans relates to footwear with a built-in scale. The described footwear with built-in scale includes a shoe, which has a means for the wearer to determine and monitor his or her body weight by way of a specially designed built-in scale. The measurement of the wearer's weight will be registered by standing still for a set period of time (usually a few seconds) with both feet parallel to one another, approximately shoulder width apart, actuating sensors placed within the weight-bearing areas of the soles of the footwear.

### SUMMARY OF THE INVENTION

The present claims provide a shoe with a scale and step counter having a transceiver for wireless communication with a smartphone or smartwatch.

Also, in some versions, the shoe has a in addition to the scale and step counter a foam wipe to clean the shoe and a magnet to hang the shoes on a magnetic surface.

And the claims provide methods of use of such shoes with a scale, step counter, wireless transceiver, foam wipe and magnet.

### BRIEF DESCRIPTION OF THE DRAWINGS

The figures which accompany the written portion of this specification illustrate versions and methods of use for the present disclosure, a shoe, constructed and operative according to the teachings of the present disclosure.

FIG. 1 is a perspective view of footwear, as described.

FIG. 2 is a top view of the footwear of FIG. 1.

FIG. 3 is a back view of the footwear of FIG. 1.

FIG. 4 is a horizontal cross-section of the footwear of FIG. 1.

FIG. 5 is a flow diagram illustrating a method of use of the footwear of FIG. 1.

### DETAILED DESCRIPTION

| | |
|---|---|
| 100 | footwear |
| 110 | shoe |
| 120 | sole |
| 210 | microcontroller |
| 220 | display |
| 230 | weight sensor |
| 240 | step sensor |
| 250 | wireless transceiver |
| 270 | magnet |
| 280 | foam wipe |
| 300 | smartphone |
| 310 | smartwatch |

As discussed above, versions of the present disclosure relate to footwear

Generally, individuals with medical conditions often need to be aware of changes in their weight. However, many individuals are either not diligent about checking their weight or do not own a scale. For some, the only time they see their weight is when they visit the hospital or doctor's office. Medical attention may be required for some individuals based on their weight. The present invention provides users with a shoe that has an integrated scale to detect weight. This offers an easy and convenient method for determining one's weight simply by wearing shoes. The shoes also send alerts to the individual's smartphone or smartwatch, indicating changes in their weight so that they may seek medical treatment if needed. This system enables individuals without a scale to check their weight with no effort and ensures that individuals with medical conditions, such as diabetes, are aware of their weight. Further, the shoes also provide for a step counter to help track the individual's movement.

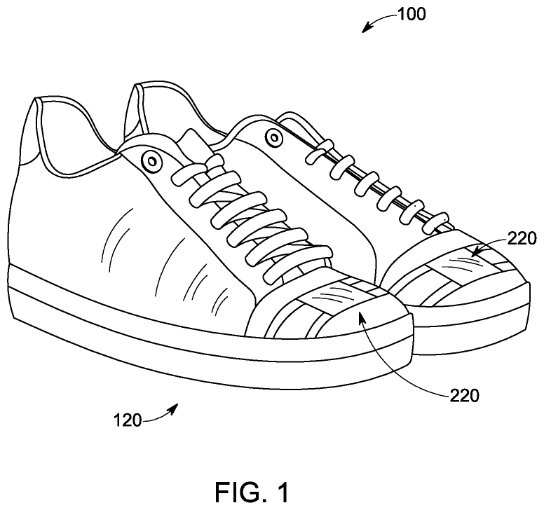

FIGS. 1-4 show various views of footwear such as shoe 100.

FIG. 1 shows a shoe 100. Here, the user may beneficially use the shoe 100 to monitor weight and step count during use. As illustrated, the shoe 100 may include a display 220 mounted to the toe of the shoe 100 and a sole 120.

FIG. 1 shows an athletic style shoe; however, any style shoe may be used.

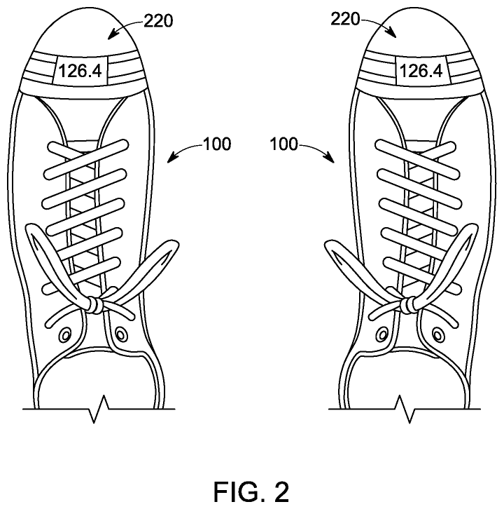

FIG. 2 shows the shoe of FIG. 1. As above, the shoe 100 may include tennis shoes, dress shoes, walking shoes, and other styles of shoes. These may use a plurality of closures such as laces, hook and loop fasteners, zippers, and other types of closures. The shoes may be made of one or more different types of material, such as cotton, polyester, leather, plastic, rubber, or other suitable materials.

The display 220 may include LCD or LED technology to present the readout from the weight sensor 230 and step sensor 240. The display 220 is 1.5 inches wide by 0.75 inches tall. Further, the display 220 may be oriented so that it is readable while the shoes are in use. The size of the screen may be changed based on the style of shoe and consumer preference.

The weight sensor 230 may be a single point load cell. The weight sensor may be situated to read even pressure across the sole for an accurate weight reading.

The step sensor 240 may be a pedometer.

The wireless transceiver may be a Bluetooth transceiver, an RF transceiver, or a WiFi transceiver. A transceiver is a device to transmit and receive data. There are both physical and wireless transceivers. Wireless transceivers are useful when, such as with shoe 100, a physical connection cannot be made. The wireless transceiver makes this possible and allows for data transmission to a smartphone or smartwatch to display at least step count and weight.

The magnet may be a ferrite, a neodymium iron boron, or a samarium cobalt magnet. The magnet may allow for interaction with magnetic surfaces outside of the heel, but not be large enough to interfere with the microcontroller.

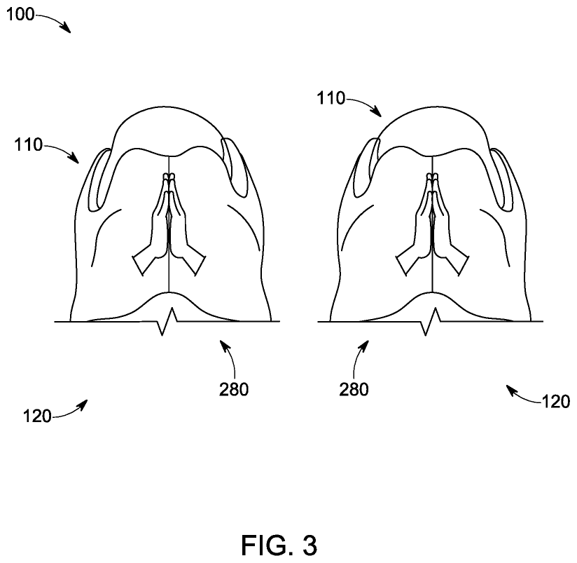

FIG. 3 is a back view of the shoe of FIG. 1. As illustrated, the footwear 100 may have a foam wipe 280 removably attached to the shoes 100. The foam wipe 280 may be useful to clean the shoes 100.

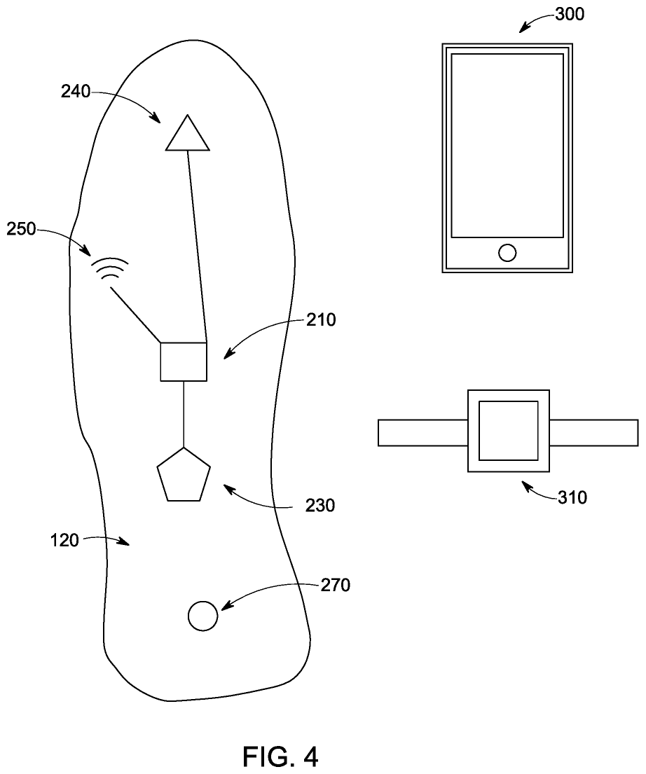

FIG. 4 is a horizontal cross-section of the sole of the shoe 100, shown in FIG. 1. In some versions, FIG. 4 represents a block-diagram version of the connectivity between the components and not an indication of the location of various components. As illustrated, the footwear 100 comprises a sole 120, including a microcontroller 210, a weight sensor 230, a step sensor 240, a wireless transceiver 250, and a magnet 270. The microcontroller 210 may be physically connected to the weight sensor 230, the step sensor 240, and the wireless transceiver 250. The present disclosure shows one microcontroller 210. However, there may be versions that have more than one microcontroller 210. In some versions, the wireless transceiver 250 wirelessly connects to a smartphone 300, or a smartwatch 310. The magnet 270 may be integrated into the heel of the shoe 100.

In some versions, the smartwatch or smartphone executes computer code that retrieves data from the shoe 100. For instance, the code sends data to the transceiver that is transferred to the microcontroller signaling the microcontroller to send data, such as weight or step data, back through the transceiver to the watch or smartphone. Alternatively, computer code executing on the microcontroller sends a signal to the smartphone or watch through the transceiver signaling the smartphone or watch that the microcontroller is ready to send weight or step data to the smartphone or watch. In some versions, the smartwatch or smartphone stores the weight or step data in the cloud.

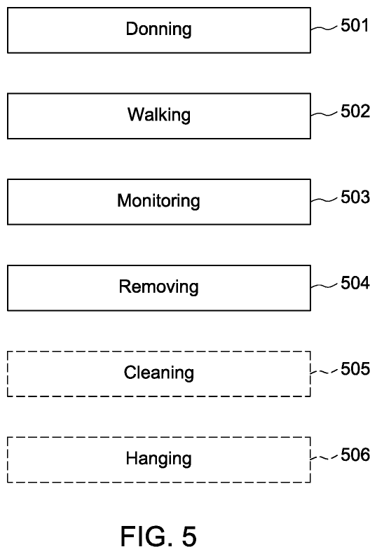

FIG. 5 is a flow diagram illustrating a method of use for the footwear. In particular, the method of use of footwear 500 may include using one or more components or features of the footwear 100, as described above. As illustrated, the method of use of the footwear 500 includes the steps of step one 501, donning shoe 100; step two 502, walking with the shoe 100 (while wearing the shoe 100); step three 503, monitoring the display 220, smartwatch 310, or smartphone 300; step four 503 removing the shoe 100; step five 503 cleaning the shoe 100 with the foam wipe 280; and step six 503 hanging the footwear with the magnet 270.

What is claimed is:

1. Footwear comprising:
a shoe having a top, a sole, a heel, and a back;
a display mounted on the top;
a weight sensor mounted in the sole;
a step sensor mounted in the sole;
and
a wireless transceiver.

2. The footwear of claim 1, wherein the heel contains a magnet.

3. The footwear of claim 1, wherein the back contains a foam wipe.

4. The footwear of claim 3, wherein the heel contains a magnet.

5. The footwear of claim 4, wherein a microcontroller connects to the display.

6. The footwear of claim 5, wherein the microcontroller connects to the weight sensor.

7. The footwear of claim 6, wherein the microcontroller connects to the step sensor.

8. The footwear of claim 7, wherein the microcontroller connects to the wireless transceiver.

10. The footwear of claim 8, wherein the wireless transceiver is a Bluetooth transceiver.

11. The footwear of claim 10, wherein the microcontroller connects to a smartwatch using the Bluetooth transceiver.

12. The footwear of claim 10, wherein the microcontroller connects to a smartphone using the Bluetooth transceiver.

9. The footwear of claim 1, wherein a microcontroller connects to the weight sensor, the step sensor, and the wireless transceiver.

13. Footwear comprising:
a shoe having a top, a sole, a heel, and a back;
a display on the top;
a weight sensor in the sole;
a step sensor in the sole;
a wireless transceiver connectable to a smartwatch or a smartphone;
a magnet in the heel;
a foam wipe in the back;
and
a microcontroller,
wherein the microcontroller connects to the display, the weight sensor, the step sensor, and the wireless transceiver.

14. A method comprising:
providing footwear comprising
a shoe having a top, a sole, a heel, and a back,
a display on the top,
a weight sensor in the sole,
a step sensor in the sole,
a wireless transceiver connectable to a smartwatch or a smartphone,
a magnet in the heel,
a foam wipe in the back,
and
a microcontroller,
wherein the microcontroller connects to the display, the weight sensor, the step sensor, and the wireless transceiver.

15. The method of claim 14 further comprising the step of donning the footwear.

16. The method of claim 15 further comprising the step of walking.

17. The method of claim 16 further comprising the step of monitoring the display, smartwatch, or smartphone.

18. The method of claim 17 further comprising the step of removing the footwear.

19. The method of claim 14 further comprising the step of cleaning the footwear with the foam wipe.

20. The method of claim 19 further comprising the step of hanging the footwear on a vertical surface with the magnet.
